(19) United States
(12) Patent Application Publication
(10) Pub. No.: US 2019/0351201 A1
(43) Pub. Date: Nov. 21, 2019

(54) HIGH-PRESSURE BALLOON CATHETERS AND METHODS

(71) Applicant: Surmodics, Inc., Eden Prairie, MN (US)

(72) Inventors: Conleth A. Mullen, Letterfrack Co. Galway (IE); Michael J. Finnerty, Ballinasloe Co. Galway (IE); Colm C. Manning, Ballinasloe Co. Galway (IE); Noel M. Gately, Ballinasloe Co. Galway (IE); Noel C. Fox, Athenry Co. Galway (IE); Nathan A. Lockwood, Minneapolis, MN (US); Joram Slager, St. Louis Park, MN (US); Amy Trocke, Plymouth, MN (US); Karl Ganske, Edina, MN (US)

(21) Appl. No.: 16/413,336

(22) Filed: May 15, 2019

Related U.S. Application Data
(60) Provisional application No. 62/672,115, filed on May 16, 2018.

(51) Int. Cl. A61M 25/10

(52) U.S. Cl. CPC A61M 25/1029; A61M 25/1002; A61M 2025/1075; A61M 25/104; A61M 25/1018

(57) ABSTRACT

Embodiments herein include high-pressure balloon catheters and methods for making the same. In an embodiment, a balloon catheter is included. The balloon catheter can include a catheter shaft and a balloon disposed on the catheter shaft. The balloon can include a wall member including an extruded material layer, a fibrous layer disposed to the outside of the extruded material layer, and at least one of a polyurethane composition and an epoxy composition contacting the fibrous layer. Other embodiments are also included herein.

This application claims the benefit of U.S. Provisional Application No. 62/672,115 filed May 16, 2018, the contents of which are herein incorporated by reference.

### FIELD

Embodiments herein relate to balloon catheters. More specifically, embodiments herein relates to high-pressure balloon catheters.

### BACKGROUND

Balloon catheters can be used for various medical purposes. Balloon catheters can be used in procedures such as renal denervation, cryoablation balloon sinuplasty, transcatheter aortic valve implantation (TAVI), AV fistula treatment, drug delivery, balloon occlusion, hypothermic cooling, ultrasonic ablation, lumbar discectomy, balloon angioplasty, esophageal dilation, atherectomy, balloon carpal tunnelplasty, kyphoplasty, and perfusion.

During use, the balloon catheter is typically moved into a desired location within a vessel and then a fluid is passed through the catheter shaft into the balloon under pressure in order to inflate the balloon. After inflation and treatment, the balloon is then deflated by letting fluid back out of the balloon and catheter can then be removed from the patient.

### SUMMARY

Embodiments herein include high-pressure balloon catheters and methods for making the same. In an embodiment, a balloon catheter is included. The balloon catheter can include a catheter shaft and a balloon disposed on the catheter shaft. The balloon can include a wall member including an extruded material layer, a fibrous layer disposed to the outside of the extruded material layer, and at least one of a polyurethane composition and an epoxy composition contacting the fibrous layer.

In an embodiment, a balloon catheter is included having a catheter shaft and a balloon disposed on the catheter shaft. The balloon can include a wall member including an inner first extruded material layer comprising a polyether block amide, an outer second extruded material layer comprising a polyamide.

In an embodiment, a balloon catheter is included having a catheter shaft and a balloon disposed on the catheter shaft. The balloon can include a wall member including an inner first extruded material layer and an outer second extruded material layer. At least one of the inner first extruded material layer and the outer second extruded material layer can include a mixture of a polyether block amide and a polyamide.

In an embodiment, a balloon catheter is included having a catheter shaft and a balloon disposed on the catheter shaft. The balloon can include a wall member including an extruded material layer including a mixture of a polyether block amide and a polyamide.

In an embodiment, a balloon catheter is included having a catheter shaft and a balloon disposed on the catheter shaft. The balloon can include a wall member having an extruded material layer and a fibrous layer disposed to the outside of the extruded material layer. The fibrous layer can include fibers of a first fiber type and fibers of a second fiber type. The first fiber type is compliant fibers and the second fiber type is non-compliant fibers.

In an embodiment, a balloon catheter is included having a catheter shaft and a balloon disposed on the catheter shaft. The balloon can include a wall member including an extruded material layer, a fibrous layer disposed to the outside of the extruded material layer, a proximal shoulder zone wherein the outer diameter of the balloon increases and a distal shoulder zone wherein the outer diameter of the balloon decreases. The balloon catheter can include an additional layer of material coated over at least a portion of an outside surface of the proximal shoulder zone and the distal shoulder zone.

In an embodiment, a balloon catheter is included having a catheter shaft and a balloon disposed on the catheter shaft. The balloon can include a wall member comprising an extruded material layer including a polyether ether ketone. A fibrous layer can be disposed to the outside of the extruded material layer.

In an embodiment, a balloon catheter is included having a catheter shaft and a balloon disposed on the catheter shaft. The balloon can include a wall member including a first material layer. The first material layer can include a polymer with polymer chains oriented radially around the circumference of the balloon.

In an embodiment, a balloon catheter is included having a catheter shaft and a balloon disposed on the catheter shaft. The balloon can include a wall member including a wall member including an inner material layer, an outer material layer, and an intermediate layer disposed between the inner and outer material layers. The intermediate layer can include a reaction product of a cross-linker that bonds the inner and outer material layers together.

In an embodiment, a balloon catheter is included having a catheter shaft and a balloon disposed on the catheter shaft. The balloon can include a wall member including a polymer layer comprising a polymer and a cross-linking agent.

In an embodiment, a balloon catheter is included having a catheter shaft and a balloon disposed on the catheter shaft. The balloon can include a wall member including a first polymer layer and a second polymer layer. The second polymer layer can include a polymer and a cross-linking agent.

In an embodiment, a balloon catheter is included having a catheter shaft and a balloon disposed on the catheter shaft. The balloon can include a wall member including an extruded material layer and a fibrous layer disposed to the outside of the extruded material layer. The fibrous layer can include a non-woven fiber mat.

In an embodiment, a balloon catheter is included having a catheter shaft and a balloon disposed on the catheter shaft. The balloon can include a wall member including an extruded material layer and a fibrous layer disposed to the outside of the extruded material layer, the fibrous layer comprising carbon nanofibers.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following drawings, in which:

FIG. 1 is a schematic view of a balloon catheter in accordance with various embodiments herein.

FIG. 2 is a cross-sectional view of a balloon catheter as taken along line 2-2′ of FIG. 1.

FIG. 3 is a cross-sectional view of a portion of a wall member of a balloon catheter as taken along line 3-3′ of FIG. 2.

FIG. 4 is a cross-sectional view of a portion of a wall member of a balloon catheter in accordance with various embodiments herein.

FIG. 5 is a cross-sectional view of a portion of a wall member of a balloon catheter in accordance with various embodiments herein.

FIG. 6 is a schematic cross-sectional view of fibers which can be part of a balloon catheter in accordance with various embodiments herein.

FIG. 7 is a schematic cross-sectional view of fibers which can be part of a balloon catheter in accordance with various embodiments herein.

FIG. 8 is a schematic cross-sectional view of fibers which can be part of a balloon catheter in accordance with various embodiments herein.

FIG. 9 is a schematic cross-sectional view of fibers which can be part of a balloon catheter in accordance with various embodiments herein.

FIG. 10 is a schematic cross-sectional view of a fiber which can be part of a balloon catheter in accordance with various embodiments herein.

FIG. 11 is a schematic cross-sectional view of fibers which can be part of a balloon catheter in accordance with various embodiments herein.

FIG. 12 is schematic cross-sectional view of a fiber which can be part of a balloon catheter in accordance with various embodiments herein.

FIG. 13 is a schematic cross-sectional view of fibers which can be part of a balloon catheter in accordance with various embodiments herein.

FIG. 14 is a cross-sectional view of a portion of a wall member of a balloon catheter in accordance with various embodiments herein.

FIG. 15 is a cross-sectional view of a portion of a wall member of a balloon catheter in accordance with various embodiments herein.

FIG. 16 is a schematic view of a balloon catheter in accordance with various embodiments herein.

FIG. 17 is a schematic view of fibers being deposited onto a balloon catheter in accordance with various embodiments herein.

FIG. 18 is a schematic view of fibers being deposited onto a balloon catheter in accordance with various embodiments herein.

FIG. 19 is a schematic view of a balloon catheter in accordance with various embodiments herein.

FIG. 20 is a schematic view of materials being deposited onto a balloon catheter in accordance with various embodiments herein.

FIG. 21 is a schematic view of materials being deposited onto a balloon catheter in accordance with various embodiments herein.

FIG. 22 is a cross-sectional view of a portion of a wall member of a balloon catheter.

While embodiments are susceptible to various modifications and alternative forms, specifics thereof have been shown by way of example and drawings, and will be described in detail. It should be understood, however, that the scope herein is not limited to the particular embodiments described. On the contrary, the intention is to cover modifications, equivalents, and alternatives falling within the spirit and scope herein.

### DETAILED DESCRIPTION

Balloon catheters can be used for various medical purposes. One type of balloon catheter is a high-pressure balloon catheter. High-pressure balloons can be substantially non-elastic or low compliant and can be formed using non-compliant or low-compliant materials or combinations thereof. A high-pressure balloon catheter can be configured to expand outward to a maximum diameter in response to being filled with a fluid under high pressure. However, the high-pressure balloon can be designed so that expansion substantially stops at the maximum diameter despite high internal pressure. This is to ensure that the balloon will not continue to expand and damage or rupture the vessel into which it is expanded. The high-pressure balloon can also have substantial structural integrity such that rupture or burst is extremely unlikely under normal circumstances of use despite the presence of the high-pressure fluid. High-pressure balloon catheters herein can be filled with a fluid having a pressure (nominal) of at least about 6, 8, 10, 12, 14, 16, 18, 20, 25, 30, 35, 40, or 50 ATM, or within a range between any of the preceding pressures. High-pressure balloons herein can expand (beyond their target expanded diameter) less than 10, 8, 6, 4, 2 or 1 percent.

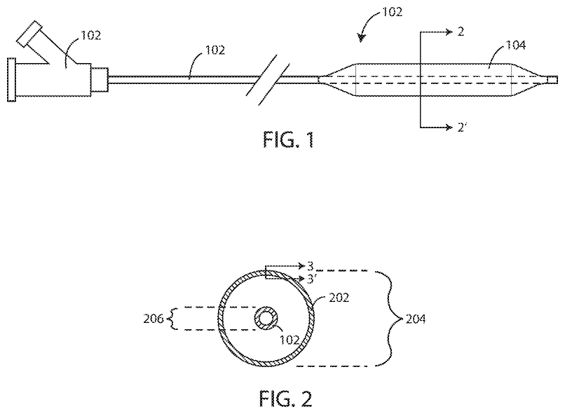

Referring now to FIG. 1, a schematic view is shown of a balloon catheter 100 in accordance with various embodiments herein. The balloon catheter 100 can include a catheter shaft 102, a balloon 104 mounted on the catheter shaft 102 near the distal end of the balloon catheter 100 and a handle and valve structure 106 disposed at the proximal end of the balloon catheter 100.

FIG. 2 is a cross-sectional view of a balloon 104 portion of a catheter 100 as taken along line 2-2′ of FIG. 1. The balloon 104 can include a wall member 202 which can form the outer diameter of the balloon 104. The balloon 104, when inflated, can have a diameter 204. The diameter 204 of the inflated balloon 104 can vary depending on the desired application. In some embodiments, the diameter 204 can be about 2, 3, 4, 5, 6, 7, 8, 9, 10, 12, 14, 16, 18, 20, 25, 30, 35, 40, or 50 millimeters, or the diameter can fall within a range wherein and of these diameters can serve as the upper or lower bound of the range. The length of the balloon can also vary. In some embodiments, the length of the balloon 104 can be about 10, 15, 20, 30, 40, 50, 60, 70, 80, 90, 100, 120, 140, 160, 180, 200, 220, 250, or 300 millimeters, or within a range between any of the preceding lengths. The diameter 206 of the catheter shaft 102 can also vary. In some embodiments, the diameter 206 can be about 1, 2, 3, 4, 5, 6, 7, 8, 9, 10, 12, or 14 millimeters or more, or within a range between any of the preceding diameters. In some embodiments, one or more lumens can be disposed within the catheter shaft 102. By way of example, the catheter shaft 102 can include a guidewire lumen and a fluid passage lumen for inflating and deflating the balloon 104. The fluid passage lumen can be in fluid communication with the interior of the balloon 104. The guidewire lumen can be sized to accommodate comment guidewire diameters including, but not limited to, 0.035″ guidewires. The overall length of the catheter shaft 102 can vary. In some embodiments, the catheter shaft 102 can be about 20, 25, 30, 35, 40, 45, 50, 55, 60, 65, 70, 75, 80, 85, or 90 centimeters in length, or can have a length falling within a range between any two of the preceding lengths.

The wall member 202 of the balloon 104 can include various layers and materials. In some embodiments, the wall member 202 can include from 1 to 10 layers. Materials of the wall member 202 can include extruded materials, fibrous materials (including woven and non-woven materials), coated layers that can be applied through spray coating, dip coating, brush coating or the like, and cross-linking layers, amongst others. The wall member 202 can be of various thicknesses. In some embodiments, the wall member 202 can be about 10, 20, 30, 40, 50, 60, 70, 80, 90, 100, 110, 120, 130, 150, 200, or 250 microns thick. In some embodiments, the thickness of the wall member 202 can fall within a range wherein any of the preceding thicknesses can serve as the upper or lower bound of the range.

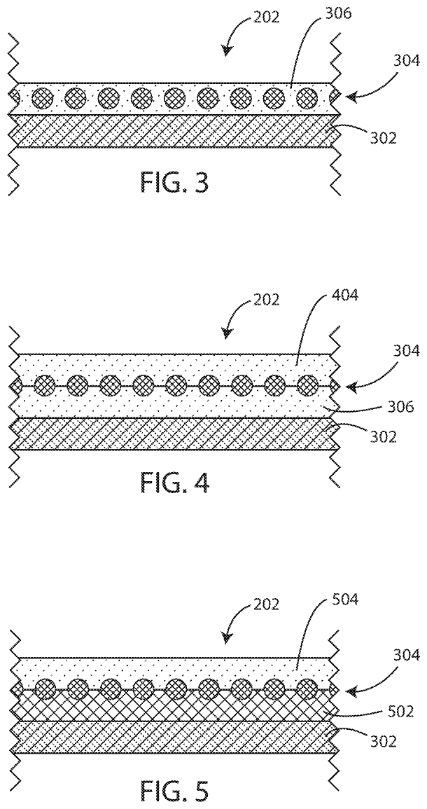

Referring now to FIG. 3, a cross-sectional view of a portion of a wall member 202 is shown as taken along line 3-3′ of FIG. 2. The wall member 202 includes a first material layer 302, that in some embodiments can be a first extruded material layer. The wall member 202 can also include a fibrous layer 304, which can be disposed over the first material layer 302. A first composition layer 306 can be disposed in contact with the fibrous layer 304. The first composition layer 306 can be above the fibrous layer 304, below the fibrous layer 304, within the fibrous layer 304, or in more than one of these positions.

Referring now to FIG. 4, a cross-sectional view of a portion of a wall member 202 is shown in accordance with various embodiments herein. The wall member 202 includes a first material layer 302, that in some embodiments can be a first extruded material layer. The wall member 202 can also include a fibrous layer 304, which can be disposed over the first material layer 302. A first composition layer 306 can be disposed in contact with the fibrous layer 304 and between the fibrous layer 304 and the first material layer 302. A second composition layer 404 can be disposed to the outside of the fibrous layer 304 and can be in contact with the fibrous layer 304. The first composition layer 306 and the second composition layer 404 can be formed of the same materials or can be formed from different materials. It will be appreciated that the presence of a material (or composition) layer over the fibrous materials can have the effect of smoothing the surface of the balloon in comparison to the level of smoothness that would be present if fibers formed the outermost surface of the balloon.

Referring now to FIG. 5, a cross-sectional view of a portion of a wall member 202 is shown in accordance with various embodiments herein. The wall member 202 includes a first material layer 302, that in some embodiments can be a first extruded material layer. The wall member 202 can also include a fibrous layer 304, which can be disposed over the first material layer 302. A first composition layer 502 can be disposed in contact with the fibrous layer 304 and between the fibrous layer 304 and the first material layer 302. A second composition layer 504 can be disposed to the outside of the fibrous layer 304 and can be in contact with the fibrous layer 304. The first composition layer 502 and the second composition layer 504 can, in this embodiment, be formed from different materials. In some embodiments, the second composition layer 504 can serve as an accessory layer. In some embodiments, the second composition layer 504 can include a polymeric matrix and an active agent. Exemplary active agents are described in greater detail below.

Fibers in the fibrous layer can take on many different configurations and patterns. By way of example, in some embodiments, fibers in the fibrous layer can be woven. In some embodiments, fibers in the fibrous layer can be non-woven and form a fibrous mat. In some embodiments, fibers in the fibrous layer can be non-woven and form a fibrous winding such that the fibers wrap around the circumference of the balloon in a helical pattern. In some embodiments, the fibrous layer can include a single of layer of fibers. In other embodiments, the fibrous layer can include multiple layers of fibers. By way of example, from 2 to 40 layers of fibers can be included.

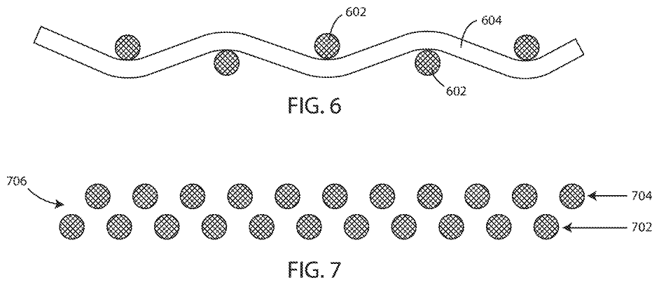

Referring now to FIG. 6, a schematic cross-sectional view of fibers is shown which can be part of a fibrous layer in a balloon catheter in accordance with various embodiments herein. In this example, the fibers can include a set of fibers 602 can are oriented in a first direction. The fibers can also include a set of fibers 604 that is oriented in a second direction. In some embodiments, the fibers can include substantially longitudinal fibers, substantially transverse fibers, or both. In some embodiments, the two sets of fibers 602, 604 can be oriented substantially perpendicular to each other. In some embodiments, the two sets of fibers 602, 604 can be oriented their intersections (or a portion of their intersections) are at an angle of about 3 degrees to 90 degrees. In some embodiments, the fibers can be woven together. Many different types of weave patterns are contemplated herein including, but not limited to, a plain weave, a satin weave, a twill weave, basket weave, rib weave, leno weave, and the like. In some embodiments, a non-stretching weave pattern can be used.

It will be appreciated that in some embodiments all of the fibers can be oriented in the same (or substantially the same) direction. In some embodiments, the fibers can be arranged as one or more layers of fibers. Referring now to FIG. 7, a schematic cross-sectional view of fibers is shown which can be part of a fibrous layer in a balloon catheter in accordance with various embodiments herein. In this example, the fibers can include a set of fibers 706 including a first fiber layer 702 and a second fiber layer 704. The number of fiber layers can be from 1 to 50 or more. In some embodiments 1, 2, 3, 4, 5, 6, 7, 8, 9, 10, 11, 12, 13, 14, or 15 fiber layers can be used. In some embodiments, a number of fiber layers can be included that falls within a range wherein the upper and lower bound of the range can be any of the preceding numbers of layers provide that the upper bound is greater than the lower bound. In this example of FIG. 7, the fibers in the different layers are all oriented in substantially the same direction. However, in some embodiments fibers in different fiber layers can be oriented in different directions.

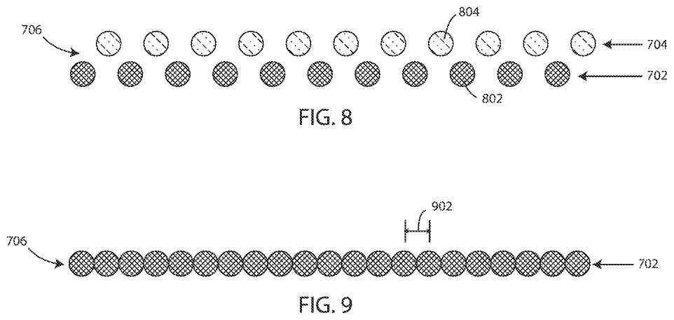

In some embodiments, all of the fibers can be of the same type. In other embodiments, multiple different types of fibers can be used, either within the same fiber layer or across different fiber layers. Referring now to FIG. 8, a schematic cross-sectional view of fibers is shown which can be part of a fibrous layer in a balloon catheter in accordance with various embodiments herein. In this example, the fibers can include a set of fibers 706 including a first fiber layer 702 and a second fiber layer 704. The fibers 802 in the first fiber layer 702 can be of a first type. The fibers 804 in the second fiber layer 804 can be of a second type. Exemplary types of fibers are described in greater detail below.

In some embodiments, fibers can be tightly packed. In some embodiments, the fibers can cover at least 50, 60, 70, 80, 90, 95, 98, or 100% of the surface area of the balloon that contains fibers. In some embodiments, the fibers can be more spaced out. In some embodiments, the fibers can cover less than or equal to 50, 40, 30, 20, 10, 5% of the surface area of the balloon that contains fibers. In some embodiments, the percent of surface area coverage of the fibers can fall within a range between any of the foregoing percentage amounts. In other embodiments, fibers can be spaced out from one another. Referring now to FIG. 9, a schematic cross-sectional view of fibers is shown which can be part of a fibrous layer in a balloon catheter in accordance with various embodiments herein. In this example, the fibers can include a set of fibers 706 including a first fiber layer 702 wherein the spacing between fibers is minimized and the average fiber pitch 902 (distance between centers of adjacent fibers) approaches the diameter of individual fibers. In other embodiments, the fibers can be more spread out from one another, such as an average fiber pitch of 1.5, 2, 3, 4, 5, 7, 10, 15, 20, 25, 30, 40, 50, 75, or 100 times the diameter of individual fibers; or the fiber pitch can be within a range wherein the upper or lower bound of the range can be selected from any of the preceding multiples of the diameter of individual fibers.

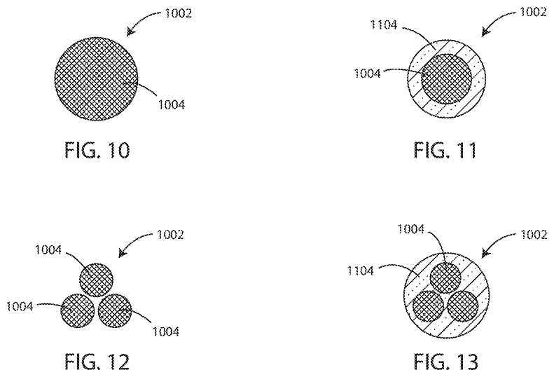

FIG. 10 is a schematic cross-sectional view of a single fiber which can be part of a balloon catheter in accordance with various embodiments herein. In this case, the fiber 1002 includes a single monofilament core 1004. In some embodiments, the fibers can be coated with another material. Referring now to FIG. 11, a schematic cross-sectional view of a fiber 1002 which can be part of a balloon catheter in accordance with various embodiments herein. In this embodiment, the fiber 1002 can include a monofilament core 1004 and a coating 1104 disposed over the monofilament core 1004. The coating 1104 can be any of various materials. In some embodiments, the coating 1104 can be an epoxy composition or a polyurethane composition such as those described herein. The coating 1104 can be formed of a material selected to provide various functionality including, but not limited to, provide adhesion, low surface energy, and the like. In some embodiments, a layer of parylene can be disposed over any of the fiber constructions described herein.

Fibers used herein can be in the form of individual filaments (e.g., a monofilament) or multifilament fibers (such as fibrous yarns, multifilament threads, or other braids that include multiple fibers). As such, general references to fibers herein can both monofilament fibers and multifilament fibers, unless the context dictates otherwise. Referring now to FIG. 12, a schematic cross-sectional view is shown of a multifilament fiber 1002 which can be part of a balloon catheter in accordance with various embodiments herein. In this example, the multifilament fiber 1002 includes three filaments 1004 which can be braided, twisted, spun or otherwise associated with one another. In some embodiments, a coating can be disposed around the multiple filaments. Referring now to FIG. 13, a schematic cross-sectional view is shown of a multifilament fiber 1002 including multiple filaments 1004 and a coating 1104 disposed around the filaments 1004. Details of exemplary coating materials for fibers are described in greater detail below.

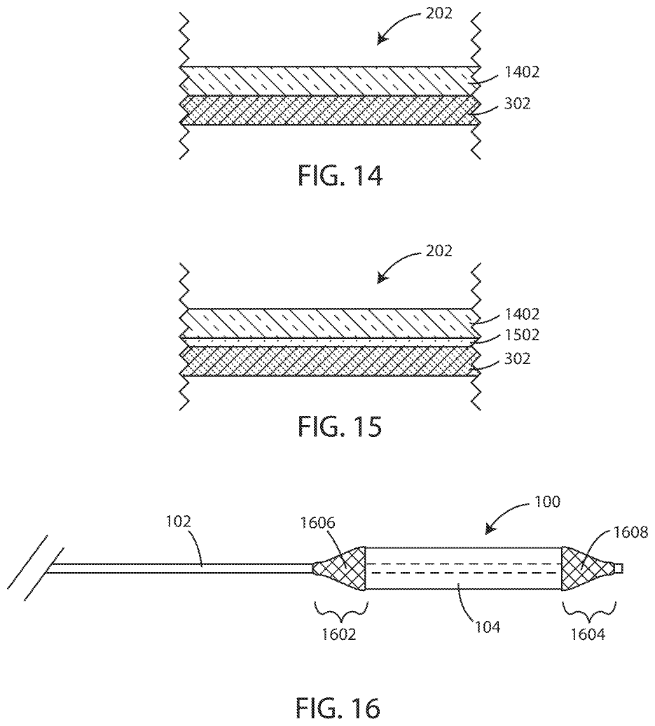

In some embodiments, walls of balloon catheters herein can include multiple extruded layers. For example, multiple materials can be coextruded together in order to create a multilayer balloon wall. Referring now to FIG. 14, a cross-sectional view is shown of a portion of a wall member 202 in accordance with various embodiments herein. The wall member 202 can include an extruded material layer 302 along with a second extruded material layer 1402 disposed on top of the extruded material layer 302. The individual extruded layers can have various thicknesses. In some embodiments, the extruded material layer 302 can be about 10, 20, 30, 40, 50, 60, 70, 80, 90, 100 or 110 microns thick, or within a range wherein the upper and lower bound can be and of the preceding provided that the upper bound is greater than the lower bound. In some embodiments, the second extruded material layer 1402 can be about 10, 20, 30, 40, 50, 60, 70, 80, 90, 100, 110 microns thick, or within a range wherein the upper and lower bound can be and of the preceding provided that the upper bound is greater than the lower bound. In some embodiments, the ratio of the thickness of extruded material layer 302 and the thickness of the second extruded material layer 1402 can be from 1:1 to 1:8, or from 1:3 to 1:5, or about 1:4.

In some embodiments, the extruded material layer 302 and the second extruded material layer 1402 can be the same material. In some embodiments, the extruded material layer 302 and the second extruded material layer 1402 can be different materials. Exemplary materials for the extruded layers are described in greater detail below. However, in some embodiments, an outer extruded layer can be more compliant than an inner extruded layer. In some embodiments, a polyamide layer can be disposed on the outside and a PEBAX layer can be disposed on the inside. However, any of the materials, including but not limited to extruded materials, described below can be on the inside, outside or a middle layer in various embodiments herein.

In some embodiments, a cross-linking layer can be disposed between layers, including but not limited to, in between extruded layers. Referring now to FIG. 15, a cross-sectional view is shown of a portion of a wall member 202 in accordance with various embodiments herein. The wall member 202 can include an extruded material layer 302 and a second extruded material layer 1402, with a cross-linking layer 1502 disposed between the extruded material layer 302 and the second extruded material layer 1402. Exemplary materials for the extruded layers are described in greater detail below. Exemplary materials for the cross-linking layer are also described in greater detail below.

In some embodiments, certain portions of the balloon can include extra material or layers in order to provide extra strength and/or different properties at those portions. For example, in some embodiments the balloon can include reinforced portions that include extra material or an extra layer. Referring now to FIG. 16, a schematic view is shown of a balloon catheter 100 in accordance with various embodiments herein. The balloon catheter 100 includes a catheter shaft 102 and a balloon 104. The balloon catheter 100 includes a proximal shoulder zone 1602 wherein the outer diameter of the balloon increases and a distal shoulder zone 1604 wherein the outer diameter of the balloon decreases. The balloon catheter 100 also include an additional segment of material 1606 deposited over at least a portion of the outside surface of the proximal shoulder zone. The balloon catheter 100 also includes an additional segment of material 1606 deposited over at least a portion of the outside surface of the distal shoulder zone. In some embodiments, the additional segment of material can include one or more layers. The additional segment of material can be spray coated, dip-coated, extruded and then fitted, wrapped on, wound on, or the like. The additional segment of material can include any of the materials of the other layers described herein above. In some embodiments, the additional segment of material can be a polyurethane or an epoxy composition described herein. In some embodiments, the additional segment of material can be an additional fibrous winding or fibrous layer such as that described for other fibrous segments herein.

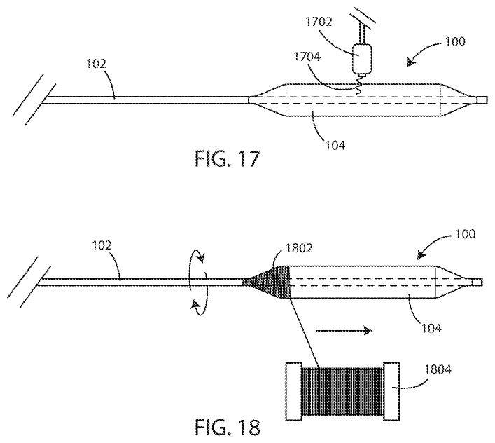

Many different techniques can be used to apply fibers and/or fibrous layers onto portions of balloon catheters herein, such as onto wall members of balloons part of balloon catheters herein. Referring now to FIG. 17, a schematic view is shown of fibers 1704 being deposited onto a balloon catheter 100 in accordance with various embodiments herein. In this view, the fibers 1704 are electrosprayed from a spray head 1702. The fibers 1704 can form an electro-sprayed non-woven fiber mat. The balloon catheter 100, including the catheter shaft 102 and the balloon 104, are stationary in some embodiments and are rotated in other embodiments during this step. In still other embodiments, the fibers may be applied to the wall member of the balloon 104 before the balloon 104 is attached to the catheter shaft 102.

In some embodiments, fibrous materials may be applied to the wall member of the balloon using a winding or wrapping technique and/or a spool-sourced technique. Fibers such as a thread or yarn can be transferred from a spool or other holding structure and then wound around the wall member of the balloon. Referring now to FIG. 18, a schematic view is shown of fibers 1802 being deposited onto a balloon catheter 100 from a spool 1804 in accordance with various embodiments herein. In some embodiments, the balloon catheter 100 can be rotated. In other embodiments, a spool 1804 or another structure such as a thread guide can be rotated around a stationary balloon catheter. In some embodiments, a single thread or yarn can be wound around the wall member of the balloon 104. In other embodiments, multiple different threads or yarns (either the same as each other or different than each other) can be simultaneously wound around the wall member of the balloon 104. Instead of simultaneously, in some embodiments, multiple different threads or yarns (either the same as each other or different than each other) can be wound around the wall member of the balloon 104 sequentially.

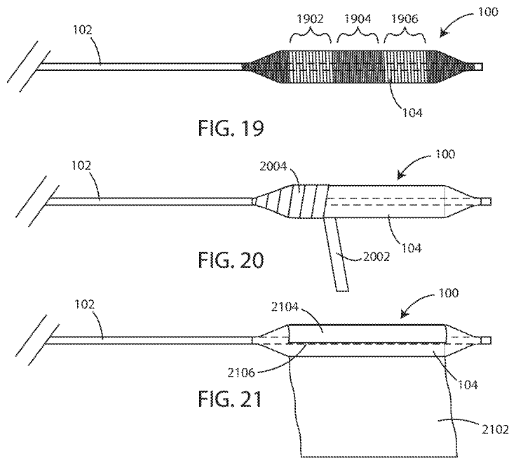

In some embodiments, the pitch of the fiber (or thread or yarn) winding can be varied such that some regions of the wall member of the balloon catheter may have a higher density of fibers than other regions. Referring now to FIG. 19, a schematic view is shown of a balloon catheter 100 in accordance with various embodiments herein. The balloon catheter 100 can include a catheter shaft 102 and a balloon 104 disposed on the catheter shaft 102. Fibers (or thread or yarn) can be disposed on the balloon 104 forming regions with different densities and/or different helical pitches. For example, the balloon 104 can include regions of lower density 1902, 1906 and regions of higher density 1904. In some embodiments, there can be from 1 to 10 regions of relatively low density and from 1 to 10 regions of relatively high density. In some embodiments, the high-density regions can have a fiber density (such as fiber count per unit length along the balloon) at least about 10, 20, 30, 50, 75, 100, 150, 200, 300, 400, or 500% higher than low density regions on the same balloon. In some embodiments, a helically arranged fibrous material can extend continuously along a segment of the balloon having a length greater than 50% of the length of the balloon.

Various compositions, including but not limited to one or more of polyurethane and epoxy compositions herein, can be applied to the balloon before, during, or after the application of the fibers over the balloon. For example, various compositions can be sprayed on, brushed on, dip-coated on, or otherwise applied. In some particular embodiments, a layer of an epoxy composition can be applied to the balloon 104 prior to application of the fibers to the balloon 104. After application of the fibers, then a layer of a polyurethane composition and/or an epoxy composition can be applied to the balloon. In various embodiments, the composition can be cured after application including, but not limited to, elevated temperature curing, UV irradiation curing, and the like.

In some embodiments, a fibrous material can be wrapped onto the balloon of a balloon catheter. In some embodiments, the fibrous material can be preformed and cut down to a desirable width for wrapping. In some embodiments, the wrapping can follow a generally helical pattern. In other embodiments, the wrapping can be non-helical (such as simply forming a circle around the balloon).

Referring now to FIG. 20, a schematic view is shown of materials being deposited onto a balloon catheter 100 in accordance with various embodiments herein. The balloon catheter 100 can include a catheter shaft 102 and a balloon 104. A fibrous material 2004 can be applied by winding or wrapping a fibrous material 2002 having a desired width around the balloon 104 can be done in a helical or non-helical fashion. In some embodiments, the leading edge of a first wrap loop meets a trailing edge of a second wrap loop that follows the first wrap loop. In some embodiments, the leading edge of a first wrap loop overlaps a trailing edge of a second wrap loop that follows the first wrap loop.

The fibrous material 2004 can be a woven or non-woven material layer. In the example of FIG. 20, the fibrous material 2002 is helically wrapped around the balloon 104. The fibrous material 2004 can be in the form of a reinforcing tape. In some embodiments, the reinforcing tape can be an aligned polymer film. In some embodiments, the reinforcing tape can be a multilayered film. In some embodiments, the reinforcing tape comprises fibers oriented along a lengthwise axis of the reinforcing tape.

Referring now to FIG. 21, a schematic view is shown of materials being deposited onto a balloon catheter 100 in accordance with various embodiments herein. The balloon catheter 100 can include a catheter shaft 102 and a balloon 104. A fibrous material 2104 can be applied by winding or wrapping a fibrous material sheet 2102 around the balloon 104 and can be done in a helical or non-helical fashion. The fibrous material 2104 can be a woven or non-woven material layer. In this example, the fibrous material sheet 2102 has a width equal to the total distance across the balloon surface for which the fibrous material will cover the balloon. In this example, the fibrous material sheet 2102 is not helically wrapped. The leading edge 2106 of the fibrous material sheet 2102 may be overlapped by the last portion (e.g., trailing edge) of the fibrous material sheet 2102 to be applied onto the balloon. However, in other embodiments, the leading edge 2106 and the trailing edge may meet each other in a flush manner such that there is no overlap and there is but a single layer of the fibrous material sheet 2102 disposed circumferentially around the wall member.

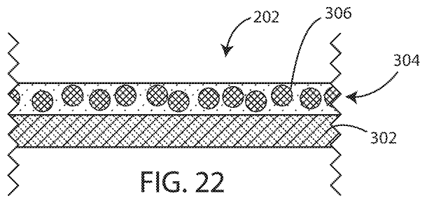

In some cases, fibers can be substantially ordered with defined and regular distances between individual fibers. However, in other embodiments, the fibers may be non-ordered. Referring now to FIG. 22, a cross-sectional view of a portion of a wall member of a balloon catheter is shown. FIG. 22 is similar to FIG. 3, however in FIG. 22 the fibers are shown disposed in a non-ordered manner.

### Polyurethane Compositions

In various embodiments, a polyurethane polymer can be used to form a portion of a balloon catheter in accordance with embodiments herein. A polyurethane is a polymer including urethane links in the polymeric chain. Polyurethane polymers can be formed by reacting a di- or poly-isocyanate with a polyol in some cases in the presence of a catalyst (such as basic and acidic amine catalysts) and/or through activation with ultraviolet light. Both the isocyanates and polyols used to make polyurethanes contain, on average, two or more functional groups per molecule. Polyurethane polymers herein can include polyurethane homopolymers, copolymers and/or terpolymers.

Many different isocyanates can be used to form polyurethanes herein. In some embodiments the isocyanate is an aromatic isocyanate. By way of example, toluene diisocyanate (TDI) and methylene diphenyl diisocyanate, MDI can be used in some embodiments.

Polyols can be of various types including, but not limited to, polyether polyols, polyester polyols, polycarbonate polyols, polycaprolactone polyols, polybutadiene polyols, and polysulfide polyols. Polyols can have varying molecular weights. In some embodiments, polyols herein can have a molecular weight of about 100 to 10,000 Daltons. A simple example of the reaction to form a polyurethane with a diol and a diisocyanate is shown below wherein R can be linear or branched alkyl, or aryl, interrupted by 0 or more heteroatoms, wherein R can be interrupted by 0 or more heteroatoms such as O, N, S, P, and Si, and wherein R′ can be C1-C30 alkyl or C5-C60 aryl.

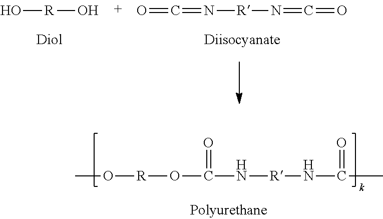

In various embodiments, chain extenders and/or cross linkers can be used during polyurethane formation. Chain extenders and cross liners are generally low molecular weight hydroxyl and/or amine terminated compounds that can substantially impact polymer morphology. Exemplary chain extenders can include difunctional hydroxide compounds (including, but not limited to, ethylene glycol, diethylene glycol, triethylene glycol, tetraethylene glycol, propylene glycol, dipropylene glycol, tripropylene glycol, 1,3-propanediol, 1,3-butanediol, 1,4-butanediol, neopentyl glycol, 1,6-hexanediol, 1,4-cyclohexanedimethanol, hydroquinone bis(2-hydroxyethyl) ether, ethanolamine, diethanolamine, methyldiethanolamine, and phenyldiethanolamine), trifunctional hydroxide compounds (including, but not limited to, glycerol, trimethylolpropane, 1,2,6-hexanetriol, triethanolamine), tetrafunctional hydroxide compounds (including but not limited to pentaerythritol, and N,N,N′,N′-Tetrakis (2-hydroxypropyl) ethylenediamine), and difunctional amine compounds (including, but not limited to, diethyltoluenediamine and dimethylthiotoluenediamine).

As such, in various embodiments herein, the polyurethane polymer can be the reaction product of an isocyanate, a polyol and a chain extender/cross linker.

In some embodiments, the polyurethane composition can be deposited from a solvent containing polyurethane composition. Aspects of solvent containing polyurethane compositions are described in US2008/0226880, the content of which is herein incorporated by reference. In some embodiments, a colloidal aqueous polyurethane dispersion can be used. Aspects of polyurethane dispersions are described in US2014/0249266 and U.S. Pat. No. 9,676,894, the content of which is herein incorporated by reference. An exemplary polyurethane dispersion is commercially available as DISPERCOLL® U from Covestro AG. In some embodiments, a self-crosslinking, aqueous polyurethane dispersion can be used. Examples of self-crosslinking, aqueous polyurethane dispersion are commercially available as WITCOBOND® W-232, W-234, W-505, W-281F, W-240, W-242, and W-244 from Chemtura.

### Epoxy Compositions

In various embodiments herein, an epoxy composition can be used. Epoxy resins, also known as polyepoxides, are a class of reactive prepolymers and polymers that contain epoxide groups. Epoxy resins can be reacted (cross-linked) either with themselves through catalytic homopolymerisation, or with a wide range of co-reactants including polyfunctional amines, acids (and acid anhydrides), phenols, alcohols and thiols.

In some embodiments, the epoxy composition can be deposited from a solvent containing epoxy composition. For example, in some embodiments an aqueous epoxy resin and an aqueous epoxy curing agent can be used. Aqueous epoxy curing agents are commercially available as ARADUR® 39, 340, 3805, 3964, 3985, 3985 S, and 3986 from Huntsman. Aqueous epoxy resins are commercially available as ARALDITE® PZ 3901, PZ 3921, PZ 3961-1, and PZ 323 from Huntsman.

### Fibers

Fibers herein can be formed of various materials. In some embodiments, fibers herein can include thermoplastic or thermoset polymers. Exemplary polymers for fibers can include, but are not limited to, polyamides (such as NYLON), polyimides, polyethylenes (including high-molecular weight polyethylene), aramids (such as) TECHNORA®), meta-aramids (such as CONEX® and ARAMET®), para-aramids (such as KEVLAR®, PARAMYD®, TWARON®), and the like. Other materials for fibers can include carbon fiber, composite fiber, and metal fiber.

As referenced above, fibers used herein can be in the form of individual filaments (e.g., a monofilament) or multifilament fibers (such as fibrous yarns, multifilament threads, or other braids that include multiple fibers). In the context of multifilament fibers or yarns, a single type of material can be used or multiple different fiber types can be spun together. In some embodiments, a blend of compliant and non-compliant fibers can be used. In some embodiments, a blend of polyamide and aramid fibers can be used. In some embodiments, a blend of polyamide and aramid fibers can be used wherein the weight ratio of polyamide to aramid fibers is from 1:20 to 20:1. In some embodiments, a blend of fibers can be annealed (heat, pressure, UV irradiation, or the like) and/or coated (such as with a UV-activated cross-linking agent or with polyurethane or epoxy compositions) after blended yarn formation to prevent separation. In some embodiments, at least one of the fibers can be thermally deformed. In some embodiments, a blend of fibers including a carbon reinforcing fiber can be used.

Fibers herein can have various diameters. However, it will be appreciated that because of the nature of fiber extrusion, there is always some fluctuation in the diameter of the filaments. As such, the measurement of denier (the weight in grams of 9000 meters of yarn) is commonly used to define the diameter. By way of example, fibers herein can be about 5, 10, 50, 100, 200, 400, 720, 1000, 1500, or 2000 denier. In some embodiments, the fiber can have a denier falling within a range wherein the upper bound and the lower bound of the range can be any of the preceding, provided that the upper bound is greater than the lower bound.

### Additional Materials

Various materials can be extruded or otherwise formed or cast in order to form one or more layers of a wall member of a balloon in accordance with embodiments herein. In some embodiments, only a single layer is extruded. In other embodiments, multiple layers are coextruded together. For example, 2, 3, 4, 5, or 6 layers can be coextruded together wherein the different layers can be different materials or the same material.

In some embodiments, the extruded material is a polymer, such as a thermoplastic polymer. To facilitate extrusion, extruded polymers typically have thermoplastic properties. Extruded materials herein can include thermoplastic homopolymers, copolymers, and terpolymers.

Polymers herein can include, but are not limited to, PET (polyethylene terephthalate), PEBA (polyether block amide, e.g. VESTAMID E or PEBAX), polyamides (such as polyamide 12, NYLON, VESTAMID), polyether ether ketone (PEEK), polyesters, polyurethanes, polyolefins, styrenic block polymers, and the like. Polymers herein can include homopolymers of the foregoing, blends including such polymers, and/or copolymers including portions of such polymers.

In some embodiments, the nylon block copolymer is a nylon block copolymer sold by ATOCHEM under the tradename PEBAX which is an elastomeric type nylon block copolymer. The commercial PEBAX polymers consist of polyether blocks separated by polyamide blocks. The polyether blocks may be based upon polyethylene glycol, polypropylene glycol, or polytetramethylene ether glycol. The polyamides are usually based upon nylon-11 but may be based upon nylons 6 of nylon-6,6 or even a copolymer such as nylon-6/nylon-11. A wide range of block polyamides have been offered and vary in the type of polyether, the nature of the polyamide block and the ratio of polyether to polyamide blocks. The polymers range in hardness from Shore A 60 to Shore D72 which is broader than for the thermoplastic polyester and thermoplastic polyurethane rubbers. Melting range is also dependent on the particular composition, and varies between 140-215 degrees Celsius.

Polymers can include polyurethanes and polyurethane copolymers, polyolefins, (including polyethylene and polypropylene), styrene-butadiene copolymers, polystyrene, polyimides, polyisoprene, isobutylene-isoprene copolymers (butyl rubber), including halogenated butyl rubber, acrylonitrile-butadiene-styrene (ABS) copolymers, silicone polymers, perfluorocarbon polymers, fluorosilicone polymers, polycarbonates, polyamides, polyesters, polyvinyl chloride, polyether-polyester copolymers, polyether-polyamide copolymers, and the like.

Polymers can also include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

In some embodiments, the polymer is selected from the group consisting of polyamide, polyimide, polyether block amide (PEBAX), polyether ether ketone (PEEK), high density polyethylene (HDPE), polyethylene, polyurethane, and polyethylene vinyl acetate.

In some embodiments an extruded substrate of a balloon, or a portion of the extruded substrate, is formed by melt extruding a thermoplastic elastomer with a vinyl pyrrolidone polymer. A “thermoplastic elastomer” (or a “thermoplastic rubber”) refers to a rubber-like material that can be processed like thermoplastic materials. Thermoplastic elastomers include copolymers and polymer blends, including those specifically described herein, having elastomeric and thermoplastic properties. Thermoplastic elastomers include styrene-based block copolymers, polyolefin polymers, thermoplastic polyurethanes, thermoplastic copolyesters, and thermoplastic polyamides, such as polyether block amide (PEBAX) polymers.

Melt extrusion can be carried out by combining raw polymeric materials including a thermoplastic elastomer, such as PEBAX, and a vinyl pyrrolidone polymer, such poly(vinyl pyrrolidone) (PVP). In some modes of practice, the extrusion uses a mixture of having an amount of vinyl pyrrolidone polymer that is lower than the amount of the thermoplastic elastomer (e.g., a low PVP/PEBAX ratio). For example, in some embodiments the vinyl pyrrolidone polymer is present in the extrusion composition in an amount of about 45% (wt.) or less, about 40% (wt.) or less, about 35% (wt.) or less, or about 30% (wt.) or less; such as in the range of about 5% (wt.) to about 45% (wt.), or about 10% (wt.) to about 40% (wt.). In some embodiments the thermoplastic elastomer, such as PEBAX, is present in the extrusion composition in an amount of about 55% (wt.) or greater, about 60% (wt.) or greater, about 65% (wt.) or greater, or about 70% (wt.) or greater; such as in the range of about 55% (wt.) to about 95% (wt.), or about 60% (wt.) to about 90% (wt.).

Melt extrusion of the polymeric materials can be performed using methods and melt extrusion equipment known in the art. For example, the polymeric starting materials, such as in the form of pellets or granules, can be fed into feeders which provide the pellets/granules into a mixing barrel having one or more heat zone(s). The melt extruder can include a screw for the heating and mixing of prior to extrusion through the die.

As described above, to facilitate extrusion, extruded polymers typically have thermoplastic properties. However, the same properties that make a material suitable for extrusion may have certain drawbacks when it comes to actual use of the medical device in practice. For example, in some cases, polymers having properties associated with thermosets can be desirable for use as materials for medical devices. As such, in accordance with embodiments herein, photoreactive cross-linking agents can be included in a composition for extrusion, but left in an unreacted state until after extrusion takes place. In this manner, desirable material properties for extrusion can be maintained and then later, after extrusion, the material properties can be changed through cross-linking in order to optimize them for use as medical devices or medical device components.

Some exemplary embodiments include the use of photoreactive crosslinking agents to convert a thermoplastic material to a thermoset material after extrusion of the thermoplastic. This conversion to a thermoset can be accomplished using actinic radiation applied to the extruded thermoplastic material, the actinic radiation activating the photoreactive crosslinking agent(s) provided in the extruded thermoplastic, thus converting it to a thermoset. Exemplary advantages of the method can include the ability to use typical and known extrusion methods for forming thermoplastic materials that include photoreactive crosslinkers in medical devices, yet provide a resulting thermoset material via post-extrusion by treatment with actinic radiation.

In some embodiments, blends including one or more materials can be used. By way of example, in some embodiments, a blend of a polyamide and a PEBAX can be used in a single extrusion layer. The extrusion can be performed producing a circular balloon substrate at the nominal expanded size of the balloon. However, various other approaches for extrusion size and geometry are also contemplated herein.

In some embodiments, such as where a blowing process is used to expand the balloon size, polymer chains of polymers that form part of balloons herein can be oriented substantially perpendicularly to the lengthwise axis of the balloon. As such, it is believed that transverse alignment of polymer chains can be used to increase the strength of the polymer resisting expansion of the balloon diameter beyond the nominal expanded diameter. In some embodiments, the average alignment of polymer chains herein is greater than 45 degrees with respect to the lengthwise axis of the balloon. In some embodiments, the average alignment of polymer chains herein is greater than 60 degrees with respect to the lengthwise axis of the balloon. In some embodiments, the polymer chains exhibit alignment with a circumferential direction around the balloon. In some embodiments, the degree of polymer chain alignment with a direction transverse to the balloon length is greater than a random orientation.

### Active Agents

In some embodiments, one or more of the layers herein (such as extruded layers, fibrous layers, polyurethane and/or epoxy layers, or the like) can include an active agent. The active agent can be configured to elute off the balloon after the balloon has been inserted into the patient transvenously.

It will be appreciated that active agents can include agents having many different types of activities. In some embodiments, active agents can include, but are not limited to, antiproliferatives such as paclitaxel, sirolimus (rapamycin), everolimus, biolimus A9, zotarolimus, tacrolimus, and pimecrolimus and mixtures thereof; analgesics and anti-inflammatory agents such as aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac; anti-arrhythmic agents such as amiodarone HCl, disopyramide, flecainide acetate, quinidine sulphate; anti-bacterial agents such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim; anti-coagulants such as dicoumarol, dipyridamole, nicoumalone, phenindione; anti-hypertensive agents such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL; anti-muscarinic agents: atropine, benzhexol HCl, biperiden, ethopropazine HCl, hyoscyamine, mepenzolate bromide, oxyphencylcimine HCl, tropicamide; anti-neoplastic agents and immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone; beta-blockers such as acebutolol, alprenolol, atenolol, labetalol, metoprolol, nadolol, oxprenolol, pindolol, propranolol; cardiac inotropic agents such as amrinone, digitoxin, digoxin, enoximone, lanatoside C, medigoxin; corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; lipid regulating agents such as bezafibrate, clofibrate, fenofibrate, gemfibrozil, probucol; nitrates and other anti-anginal agents such as amyl nitrate, glyceryl trinitrate, isosorbide dinitrate, isosorbide mononitrate, pentaerythritol tetranitrate.

Other active agents include, but are not limited to, active agents for treatment of hypertension (HTN), such as guanethidine.

In a particular embodiment, the active agent is selected from the group of paclitaxel, sirolimus (rapamycin), everolimus, biolimus A9, zotarolimus, tacrolimus, and pimecrolimus and mixtures thereof.

Active agents herein can also include nucleic acids that can function to provide a therapeutic effect. Exemplary types of nucleic acids can include, but are not limited to, ribonucleic acids (RNA), deoxyribonucleic acids (DNA), small interfering RNA (siRNA), micro RNA (miRNA), piwi-interacting RNA (piRNA), short hairpin RNA (shRNA), antisense nucleic acids, aptamers, ribozymes, locked nucleic acids and catalytic DNA.

### Cross-Linking Materials

Various cross-linking materials can be used with embodiments herein. In some embodiments, cross-linking materials may be applied as a distinct layer between two other material layers. In other embodiments, cross-linking materials can be mixed or otherwise blended in with materials of other layers.

In some embodiments, cross-linking agents used in accordance with embodiments herein can include those with at least two photoreactive groups. Exemplary cross-linking agents are described in U.S. Publ. Pat. App. No. 2011/0245367, the content of which is herein incorporated by reference in its entirety.

In some embodiments, the first and/or second crosslinking agent can have a molecular weight of less than about 1500 kDa. In some embodiments, the crosslinking agent can have a molecular weight of less than about 1200, 1100, 1000, 900, 800, 700, 600, 500, or 400.

In some embodiments, at least one of the first and second cross-linking agents comprising a linking agent having formula Photo¹-LG-Photo², wherein Photo¹ and Photo², independently represent at least one photoreactive group and LG represents a linking group comprising at least one silicon or at least one phosphorus atom, there is a covalent linkage between at least one photoreactive group and the linking group, wherein the covalent linkage between at least one photoreactive group and the linking group is interrupted by at least one heteroatom.

In some embodiments, at least one of the first and second cross-linking agents comprising a linking agent having a formula selected from:

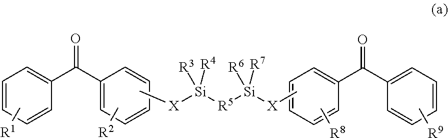

wherein R1, R2, R8 and R9 are H, methyl, ethyl, or any substitution; R3, R4, R6 and R7 are alkyl, aryl, or a combination thereof; R5 is C1-C20 alkyl or aryl or any substitution; and each X, independently, is O, N, Se, S, or alkyl, or a combination thereof;

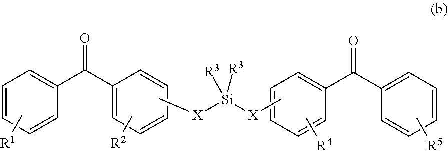

wherein R1, R2, R4, R5 are H, methyl, ethyl, or any substitution; R3 can be alkyl, aryl, or a combination thereof; and X, independently, are O, N, Se, S, alkylene, or a combination thereof;

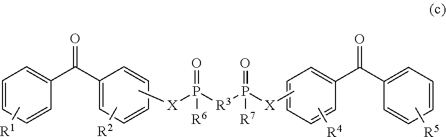

wherein R1, R2, R4 and R5 are H, methyl, ethyl, or any substitution; R3 is C1-C20 alkyl or aryl or any substitution; R6 and R7 are alkyl, aryl, or a combination thereof; and each X can independently be O, N. Se, S, alkylene, or a combination thereof; and

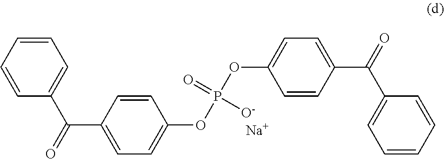 [SMILES: O=C(c1ccccc1)c1ccc(OP(=O)([O-])Oc2ccc(C(=O)c3ccccc3)cc2)cc1 ; [Na+]]

In a particular embodiment, the cross-linking agent can be bis(4-benzoylphenyl) phosphate.

In some embodiments, the photoactivatable cross-linking agent can be ionic, and can have good solubility in an aqueous composition, such as the first and/or second coating composition. Thus, in some embodiments, at least one ionic photoactivatable cross-linking agent is used to form the coating. In some cases, an ionic photoactivatable cross-linking agent can crosslink the polymers within the second coating layer which can also improve the durability of the coating.

Any suitable ionic photoactivatable cross-linking agent can be used. In some embodiments, the ionic photoactivatable cross-linking agent is a compound of formula I: X₁-Y-X₂ where Y is a radical containing at least one acidic group, basic group, or a salt of an acidic group or basic group. X₁ and X₂ are each independently a radical containing a latent photoreactive group. The photoreactive groups can be the same as those described herein. Spacers can also be part of X₁ or X₂ along with the latent photoreactive group. In some embodiments, the latent photoreactive group includes an aryl ketone or a quinone.

The radical Y in formula I provides the desired water solubility for the ionic photoactivatable cross-linking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable cross-linking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic cross-linking agent includes a sulfonic acid or sulfonate group. Suitable counter ions include alkali, alkaline earths metals, ammonium, protonated amines, and the like.

For example, a compound of formula I can have a radical Y that contains a sulfonic acid or sulfonate group; X₁ and X₂ can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X₁ and X₂ can contain photoreactive groups that include aryl ketones. Such photoactivatable cross-linking agents include ethylenebis(4-benzoylbenzyldimethylammonium) salt; hexamethylenebis (4-benzoylbenzyldimethylammonium) salt; 1,4-bis(4-benzoylbenzyl)-1,4-dimethylpiperazinediium) salt, bis(4-benzoylbenzyl)hexamethylenetetraminediium salt, bis[2-(4-benzoylbenzyldimethylammonio)ethyl]-4-benzoylbenzylmethylammonium salt; 4,4-bis(4-benzoylbenzyl)morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl)piperzinediium salt. See U.S. Pat. No. 5,714,360. The counter ion is typically a carboxylate ion or a halide. On one embodiment, the halide is bromide.

In other embodiments, the ionic photoactivatable cross-linking agent can be a compound having the formula:

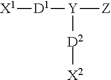

wherein X¹ includes a first photoreactive group; X² includes a second photoreactive group; Y includes a core molecule; Z includes at least one charged group; D¹ includes a first degradable linker; and D² includes a second degradable linker. Additional exemplary degradable ionic photoactivatable cross-linking agents are described in US Patent Application Publication US 2011/0144373 (Swan et al., “Water Soluble Degradable Crosslinker”), the disclosure of which is incorporated herein by reference.

In some aspects a non-ionic photoactivatable cross-linking agent can be used. In one embodiment, the non-ionic photoactivatable cross-linking agent has the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

In other embodiments, the non-ionic photoactivatable cross-linking agent can be represented by the formula:

PG²-LE²-X-LE¹-PG¹

wherein PG¹ and PG² include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE¹ and LE² are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof; or a combination thereof. Other non-ionic crosslinking agents are described, for example, in U.S. application Ser. No. 13/316,030 filed Dec. 9, 2011 (Publ. No. US 2012/0149934) (Kurdyumov, “Photocrosslinker”), the disclosure of which is incorporated herein by reference.

Further embodiments of non-ionic photoactivatable cross-linking agents can include, for example, those described in U.S. Provisional Application 61/494,724 filed Jun. 8, 2011 (now U.S. application Ser. No. 13/490,994) (Swan et al., “Photo-Vinyl Primers/Crosslinkers”), the disclosure of which is incorporated herein by reference. Exemplary cross-linking agents can include non-ionic photoactivatable cross-linking agents having the general formula R¹—X—R², wherein R¹ is a radical comprising a vinyl group, X is a radical comprising from about one to about twenty carbon atoms, and R² is a radical comprising a photoreactive group.

Some suitable cross-linking agents are those formed by a mixture of the chemical backbone molecule (such as pentaerythritol) and an excess of a derivative of the photoreactive group (such as 4-bromomethylbenzophenone). An exemplary product is tetrakis(4-benzoylbenzyl ether) of pentaerythritol (tetrakis(4-benzoylphenylmethoxymethyl)methane). See U.S. Pat. Nos. 5,414,075 and 5,637,460.

A single photoactivatable cross-linking agent or any combination of photoactivatable cross-linking agents can be used in forming the coating. In some embodiments, at least one nonionic cross-linking agent such as tetrakis(4-benzoylbenzyl ether) of pentaerythritol can be used with at least one ionic cross-linking agent. For example, at least one non-ionic photoactivatable cross-linking agent can be used with at least one cationic photoactivatable cross-linking agent such as an ethylenebis(4-benzoylbenzyldimethylammonium) salt or at least one anionic photoactivatable cross-linking agent such as 4,5-bis(4-benzoyl-phenylmethyleneoxy)benzene-1,3-disulfonic acid or salt. In another example, at least one nonionic cross-linking agent can be used with at least one cationic cross-linking agent and at least one anionic cross-linking agent. In yet another example, a least one cationic cross-linking agent can be used with at least one anionic cross-linking agent but without a non-ionic cross-linking agent.

An exemplary cross-linking agent is disodium 4,5-bis[(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

A further exemplary cross-linking agent is ethylenebis (4-benzoylbenzyldimethylammonium) dibromide. This agent can be prepared as described in U.S. Pat. No. 5,714,360, the content of which is herein incorporated by reference.

Further cross-linking agents can include the cross-linking agents described in U.S. Publ. Pat. App. No. 2010/0274012 and U.S. Pat. No. 7,772,393 the content of all of which is herein incorporated by reference.

In some embodiments, cross-linking agents can include boron-containing linking agents including, but not limited to, the boron-containing linking agents disclosed in U.S. 61/666,516, entitled “Boron-Containing Linking Agents” by Kurdyumov et al., the content of which is herein incorporated by reference. By way of example, linking agents can include borate, borazine, or boronate groups and coatings and devices that incorporate such linking agents, along with related methods. In an embodiment, the linking agent includes a compound having the structure (I):

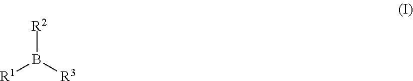

wherein R¹ is a radical comprising a photoreactive group; R² is selected from OH and a radical comprising a photoreactive group, an akyl group and an aryl group; and R³ is selected from OH and a radical comprising a photoreactive group. In some embodiments the bonds B—R¹, B—R² and B—R³ can be chosen independently to be interrupted by a heteroatom, such as O, N, S, or mixtures thereof.

Additional agents for use with embodiments herein can include stilbene-based reactive compounds including, but not limited to, those disclosed in U.S. 61/736,436, entitled “Stilbene-Based Reactive Compounds, Polymeric Matrices Formed Therefrom, and Articles Visualizable by Fluorescence” by Kurdyumov et al., the content of which is herein incorporated by reference.

Additional photoreactive agents, cross-linking agents, and associated reagents are disclosed in US2011/0059874; US 2011/0046255; and US 2010/0198168, the content of all of which is herein incorporated by reference.

### Photoreactive Groups

Various components used in compositions herein can include photoreactive groups. As used herein, the phrases “latent photoreactive group” and “photoreactive group” are used interchangeably and refer to a chemical moiety that is sufficiently stable to remain in an inactive state (i.e., ground state) under normal storage conditions but that can undergo a transformation from the inactive state to an activated state when subjected to an appropriate energy source. Unless otherwise stated, references to photoreactive groups herein shall also include the reaction products of the photoreactive groups. Photoreactive groups respond to specific applied external stimuli to undergo active specie generation with resultant covalent bonding to an adjacent chemical structure. For example, a photoreactive group can be activated and can abstract a hydrogen atom from an alkyl group. A covalent bond can then form between the compound with the photoreactive group and the compound with the C—H bond. Suitable photoreactive groups are described in U.S. Pat. No. 5,002,582, the disclosure of which is incorporated herein by reference.

Photoreactive groups can be chosen to be responsive to various portions of actinic radiation. For example, groups can be chosen that can be photoactivated using either ultraviolet or visible radiation. Suitable photoreactive groups include, for example, azides, diazos, diazirines, ketones, and quinones. The photoreactive groups generate active species such as free radicals including, for example, nitrenes, carbenes, and excited states of ketones upon absorption of electromagnetic energy.

The photoreactive group can comprise an aryl ketone, such as acetophenone, benzophenone, anthrone, and anthrone-like heterocycles (i.e., heterocyclic analogs of anthrone such as those having N, O, or S in the 10-position), or their substituted (e.g., ring substituted) derivatives. Examples of aryl ketones include heterocyclic derivatives of anthrone, including acridone, xanthone, and thioxanthone, and their ring substituted derivatives. Other suitable photoreactive groups include quinone such as, for example anthraquinone.

The functional groups of such aryl ketones can undergo multiple activation/inactivation/reactivation cycles. For example, benzophenone is capable of photochemical excitation with the initial formation of an excited singlet state that undergoes intersystem crossing to the triplet state. The excited triplet state can insert into carbon-hydrogen bonds by abstraction of a hydrogen atom (from a polymeric coating layer, for example), thus creating a radical pair. Subsequent collapse of the radical pair leads to formation of a new carbon-carbon bond. If a reactive bond (e.g., carbon/hydrogen) is not available for bonding, the ultraviolet light-induced excitation of the benzophenone group is reversible and the molecule returns to ground state energy level upon removal of the energy source. Photoreactive aryl ketones such as benzophenone and acetophenone can undergo multiple reactivations in water and hence can provide increased coating efficiency.

The azides constitute another class of photoreactive groups and include arylazides (C₆R₅N₃) such as phenyl azide and 4-fluoro-3-nitrophenyl azide; acyl azides (—CO—N₃) such as benzoyl azide and p-methylbenzoyl azide; azido formates (—O—CO—N₃) such as ethyl azidoformate and phenyl azidoformate; sulfonyl azides (—SO₂—N₃) such as benzenesulfonyl azide; and phosphoryl azides (RO)₂PON₃ such as diphenyl phosphoryl azide and diethyl phosphoryl azide.

Diazo compounds constitute another class of photoreactive groups and include diazoalkanes (—CHN₂) such as diazomethane and diphenyldiazomethane; diazoketones (—CO—CHN₂) such as diazoacetophenone and 1-trifluoromethyl-1-diazo-2-pentanone; diazoacetates (—O—CO—CHN₂) such as t-butyl diazoacetate and phenyl diazoacetate; and beta-keto-alpha-diazoacetates (—CO—CN₂—CO—O—) such as t-butyl alpha diazoacetoacetate.

Other photoreactive groups include the diazirines (—CHN₂) such as 3-trifluoromethyl-3-phenyldiazirine; and ketenes (—CH═C═O) such as ketene and diphenylketene.

In some aspects, the photoreactive groups can be aryl ketones, such as benzophenone.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

Aspects have been described with reference to various specific and preferred embodiments and techniques. However, it should be understood that many variations and modifications may be made while remaining within the spirit and scope herein. As such, the embodiments described herein are not intended to be exhaustive or to limit the invention to the precise forms disclosed in the following detailed description. Rather, the embodiments are chosen and described so that others skilled in the art can appreciate and understand the principles and practices.

1. A balloon catheter comprising
a catheter shaft; and
a balloon disposed on the catheter shaft, the balloon comprising
a wall member, the wall member comprising
an extruded material layer;
a fibrous layer disposed to the outside of the extruded material layer; and
at least one of a polyurethane composition and an epoxy composition contacting the fibrous layer.

2. The balloon catheter of claim 1, the polyurethane composition binding the fibrous layer to the extruded material layer.

3. The balloon catheter of claim 1, the polyurethane composition comprising an aqueous polyurethane dispersion.

4. The balloon catheter of claim 1, the polyurethane comprising a cross-linkable polyurethane.

5. The balloon catheter of claim 1, the fibrous layer comprising braided yarn.

6. The balloon catheter of claim 5, the polyurethane composition surrounding the yarn within the fibrous layer.

7. The balloon catheter of claim 1, the epoxy composition binding the fibrous layer to the extruded material layer.

8. The balloon catheter of claim 1, the epoxy composition surrounding a yarn within the fibrous layer.

9. The balloon catheter of claim 5, the braided yarn comprising fibers comprising a material selected from the group consisting of aromatic aramid, polyethylene, and polyamide.

10. The balloon catheter of claim 1, the wall member further comprising a second extruded material layer.

11. A balloon catheter comprising
a catheter shaft; and
a balloon disposed on the catheter shaft, the balloon comprising
a wall member, the wall member comprising
an inner first extruded material layer comprising a polyether block amide;
an outer second extruded material layer comprising a polyamide.

12. The balloon catheter of claim 11, the inner first extruded material layer having a first thickness, the outer second extruded material layer comprising a second thickness, the ratio of the first thickness to the second thickness from 1:1 to 1:8.

13. The balloon catheter of claim 12, the ratio of the first thickness to the second thickness being from 1:3 to 1:5.

14. The balloon catheter of claim 12, the ratio of the first thickness to the second thickness being 1:4.

15. A balloon catheter comprising
a catheter shaft; and
a balloon disposed on the catheter shaft, the balloon comprising
a wall member, the wall member comprising
an extruded material layer;
a fibrous layer disposed to the outside of the extruded material layer;
the fibrous layer comprising fibers of a first fiber type and fibers of a second fiber type, wherein the first fiber type is compliant fibers and the second fiber type is non-compliant fibers.

16. The balloon catheter of claim 15, the first fiber type comprising polyamide fibers.

17. The balloon catheter of claim 15, the first fiber type comprising para-aramid fibers.

18. The balloon catheter of claim 15, wherein the fibers of the first fiber type and the fibers of the second fiber type are braided.

19. The balloon catheter of claim 15, wherein the fibers of the first fiber type and the fibers of the second fiber type are annealed together.

20. The balloon catheter of claim 15, wherein the fibers of the first fiber type are thermally deformed.
